(19) United States
(12) Patent Application Publication
(10) Pub. No.: US 2025/0268682 A1
(43) Pub. Date: Aug. 28, 2025

(54) SKULL CLAMP WITH SAGITTAL ADJUSTMENT

(71) Applicant: pro med instruments GmbH, Freiburg im Breisgau (DE)

(72) Inventors: Valentina Tollinger, Freiburg im Breisgau (DE); Severin Moosmann, Friesenheim (DE); Andreas Blum, Ehrenkirchen (DE); Simon-Martin Hofer, Appenweier (DE); Christian Schlenk, Oberkirch (DE)

(21) Appl. No.: 19/066,305

(22) Filed: Feb. 28, 2025

Related U.S. Application Data
(60) Provisional application No. 63/559,118, filed on Feb. 28, 2024.

(51) Int. Cl. A61B 90/14

(52) U.S. Cl. CPC A61B 90/14

(57) ABSTRACT

A head fixation device in the form of a skull clamp includes an adjustment assembly along a base of the skull clamp. The adjustment assembly includes one or more locking features that place the skull clamp in an adjustable or fixed state. In the adjustable state the skull clamp is adjustable about an axis defined by the base which is perpendicular to the sagittal plane. In this manner a portion of the skull clamp is movable along the sagittal plane and such adjustment allows the base of the skull clamp to maintain its relative position to a table adapter and/or patient support table. The device includes a positioning adapter that connects the skull clamp with a table adapter. The positioning adapter includes an adjustable degree of freedom for the skull clamp to permit rotation of the skull clamp about an axis perpendicular to the axis defined by the base.

### PRIORITY

This application claims priority to U.S. Provisional Patent Application 63/559,118 entitled “Skull Clamp with Sagittal Adjustment,” filed Feb. 28, 2024, which is hereby incorporated by reference.

### BACKGROUND

This disclosure relates to the medical field for head and neck procedures including surgery and imaging, and pertaining particularly to certain medical devices used to stabilize a patient for such procedures. In such procedures a patient can be positioned on a table or board structure (e.g., surgical table, OR table, transport table, transfer board, etc.) and then stabilized using a device such as a head fixation device, sometimes in the form of a skull clamp. Where imaging procedures are involved, a stabilized patient can be positioned within the gantry of an MRI machine. In some instances, MRI headcoils can be positioned about the patient's stabilized head to acquire head and/or neck images. While a variety of headrest systems have been made and used, it is believed that no one prior to the inventor(s) has made or used an invention as described herein.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements.

FIG. 1A depicts a top perspective view of an exemplary skull clamp connected with a positioning adapter.

FIG. 1B depicts a front view of the skull clamp and positioning adapter of FIG. 1A, shown with stabilization or contact features in the form of skull pins with various axes defined by the stabilization or contact features.

FIG. 2 depicts a side view of the skull clamp and positioning adapter of FIG. 1A, shown in a first rotatable position about an axis defined by the skull clamp base.

FIG. 3 depicts a top perspective view of a table adapter that is connectable with the positioning adapter of FIG. 1A to connect the skull clamp and positioning adapter with a patient support table or structure.

FIG. 4 depicts a top perspective cross section view of an interface between the skull clamp and the positioning adapter, shown with the skull clamp in an adjustable state.

FIG. 5 depicts a top perspective cross section view of an interface between the skull clamp and the positioning adapter, shown with the skull clamp in a fixed state.

FIG. 6 depicts a top perspective cross section view of an interface between the skull clamp and the positioning adapter, shown with the skull clamp in an adjustable state and showing a lever for disconnecting the skull clamp from the positioning adapter.

FIG. 7 depicts a top perspective view of the skull clamp and positioning adapter of FIG. 1A with the skull clamp adjusted to an alternate rotational position.

FIG. 8 depicts a side view of the skull clamp and positioning adapter of FIG. 7, shown in a second rotatable position about an axis defined by the skull clamp base.

FIG. 9 depicts a perspective cross section view of an adjustment assembly that permits rotation of the skull clamp between the first and second rotatable positions shown in FIGS. 2 and 8.

FIG. 10 depicts an anatomical illustration of a patient and the planes defined by the body of the patient.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples should not be used to limit the scope of protection provided by this document or any related document. Other examples, features, aspects, embodiments, and advantages of the disclosed technology will become apparent to those skilled in the art from the following description, which is by way of illustration, one of the best modes contemplated for making and using embodiments of the disclosed technology. As will be realized, the disclosed technology is capable of other different and obvious implementations. Accordingly, the drawings and descriptions should be regarded as illustrative in nature and not restrictive.

It is understood that any one or more of the teachings, expressions, versions, examples, etc. described herein may be combined with any one or more of the other teachings, expressions, versions, examples, etc. that are described herein. The following-described teachings, expressions, versions, examples, etc. should therefore not be viewed in isolation relative to each other. Various suitable ways in which the teachings herein may be combined will be readily apparent to those skilled in the art in view of the teachings herein. Such modifications and variations are intended to be included within the scope of the claims.

When stabilizing patients for head, neck, or other procedures that may be surgical or diagnostic, it is desirable to provide the users of the stabilization equipment with equipment and ways to adjust the stabilization equipment. For instance, adjustability can be desired to achieve a desired patient stabilization position that is considered well suited or optimal for a particular procedure. In some cases, the procedure could be a surgical procedure or it could be an imaging procedure.

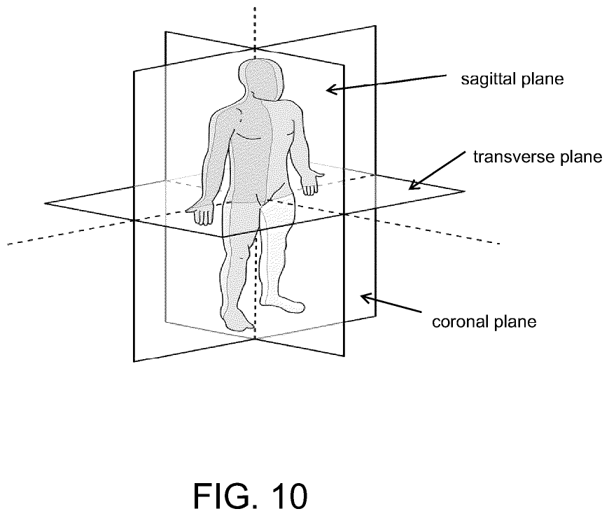

With prior art head stabilization equipment some adjustment degrees of freedom were limited, or they required complex or multiple steps to adjust or reposition the head stabilization equipment. In one such example, tilting a skull clamp along a sagittal plane (refer to FIG. 10) required changing the location of the base of the skull clamp relative to the patient support table. This made it difficult or impossible to achieve certain positionings as the base of the skull clamp would contact the table and thereby be prevented from further adjustment in that manner due to the interference or obstruction.

Even where certain adjustment ranges could be achieved, in intraoperative procedures involving MRI scanners, it was difficult or impossible to maintain the patient's head being imaged in the isocenter of the MRI scanner to get the best imaging output. For example, with the prior art skull clamps it was not feasible to conduct procedures using intraoperative scanners that required access to the patient's frontal temporal position. Additionally, some other procedures, such as posterior cervical fusion procedures, were difficult to execute using an intraoperative scanner. As the sophistication of procedures continues to progress, the demand for image-guided procedures is increasing. To support these procedures that use or want to use intraoperative MRI scanners, new stabilization equipment features like those described, shown, and claimed here are needed.

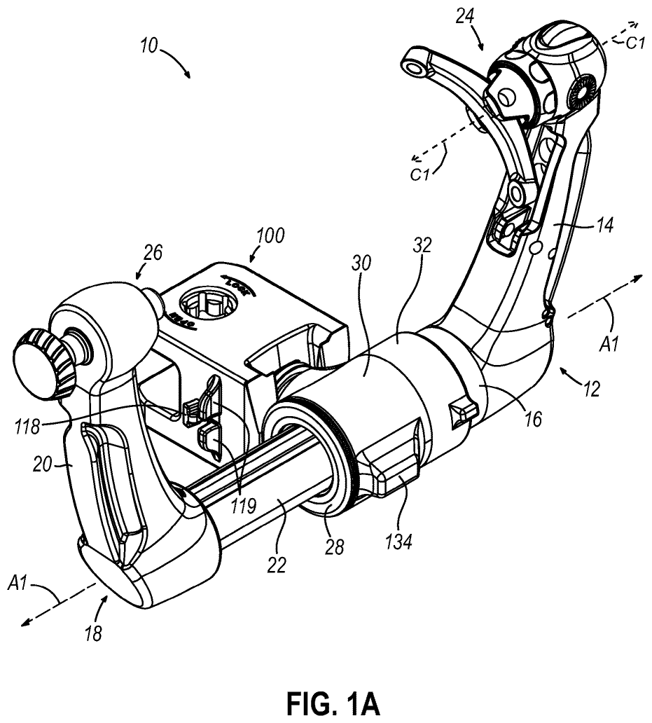

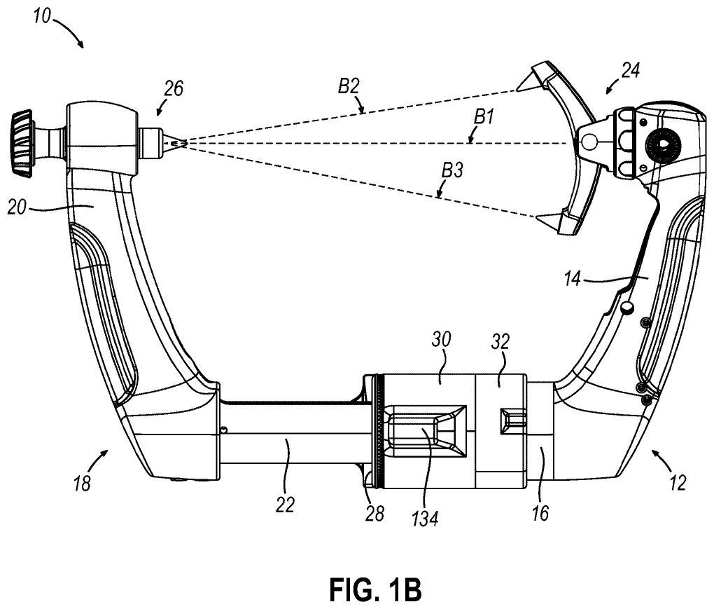

FIGS. 1A and 1B illustrates a skull clamp (10) connected with a positioning adapter (100). The skull clamp (100) includes a first arm (12) having an upright portion (14) and a lateral portion (16), and a second arm (18) having an upright portion (20) and a lateral portion (22). The skull clamp (100) also includes a 2-pin stabilization assembly (24) connected with an end of the upright portion (14) of the first arm (12), and a single pin stabilization assembly (26) connected with an end of the upright portion (20) of the second arm (18). The 2-pin stabilization assembly (24) is rotatable about a central axis (C1) as can be seen in FIG. 1A and by comparing the position of the 2-pin stabilization assembly in FIG. 1A and FIG. 1B. The stabilization assemblies (24, 26) are configured to retain skull pins (refer to FIG. 1B), which are configured to contact a head of a patient to stabilize the head of the patient. To accommodate various patient head sizes and patient positions for stabilization, the first and second arms (12, 18) are adjustable to alter the width between the stabilization assemblies (24, 26). In some examples the adjustment of the width between the stabilization assemblies (24, 26) uses ratcheting features within the lateral portions (16, 22) of the first and second arms (12, 18). In some other examples this adjustment of the width moves the lateral portions (16, 22) telescopically relative to one another. As shown in FIG. 1, the combined lateral portions (16, 22) of the first and second arms (12, 18) define a base and further define an axis (A1). As will be discussed further below, the skull clamp (10) also includes a toothed ring (28) or first locking feature, a connection member (30) or second locking feature, and a lock ring (32) or third locking feature.

FIG. 1B includes various axes that are defined by components or features of the skull clamp (10). For instance, each of the contact features or stabilization features, here represented as skull pins, define a respective longitudinal axis (B1, B2, B3) that extends through a distal end of the skull pin which is configured to contact the head of the patient. In the present example, this arrangement of the three contact features is such that they collectively define an isosceles triangle where the isosceles triangle is divided by the longitudinal axis (B1) of the single contact feature. In combination with the rotatability of the 2-pin stabilization assembly (24), this defined isosceles triangle can be considered rotatable also. In this regard the isosceles triangle is rotatable about the axis (B1) defined by the single contact feature or skull pin.

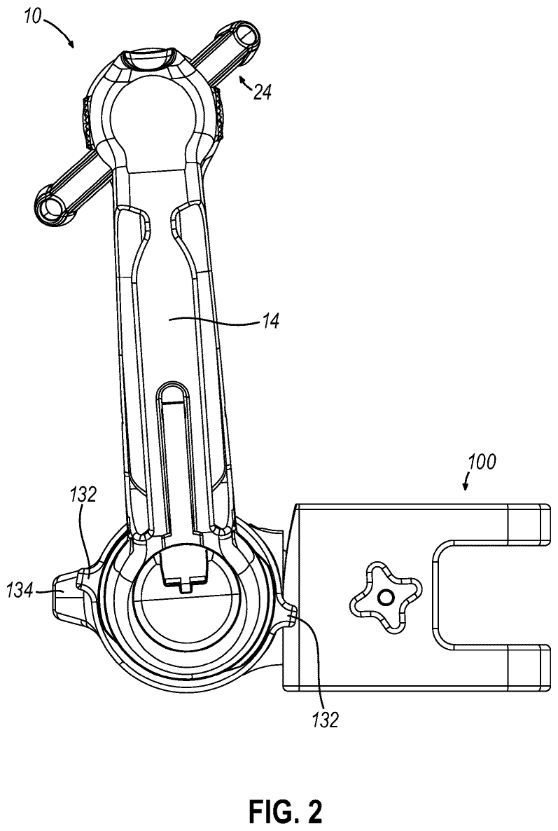

Referring to FIG. 2, the skull clamp (10) and position adapter (100) are shown in a connected and fixed or locked state with the skull clamp (10) positioned at a first rotational position or state. As will be described in greater detail below, skull clamp (10) may be rotatably adjustable about the axis (A1) defined by the lateral portions (16, 22) of the skull clamp (10), which may also be referred to as the base of the skull clamp (10). Additionally, when adjusting the skull clamp (10) in this manner the connection of the skull clamp (10) with the positioning adapter (100) is maintained.

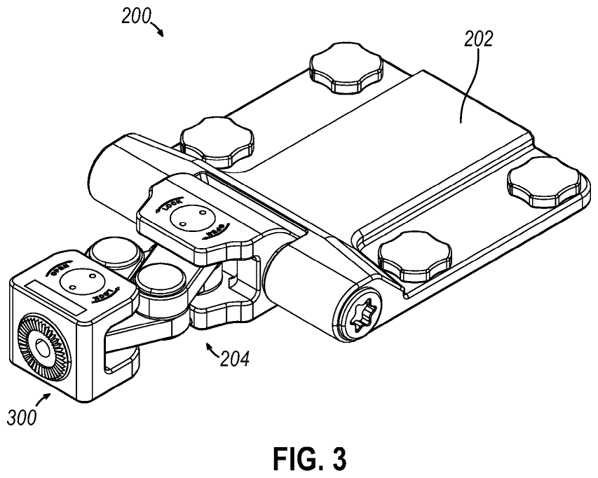

Referring to FIG. 3, a table adapter (200) is shown, which includes a first portion (202) that is configured to connect with a patient support structure (not shown). At the other end of the table adapter (200) a positioning adapter (300) connects with a joint assembly (204). Table adapter (200), positioning adapter (300), and joint assembly (204) are described in greater detail in U.S. Pat. No. 9,216,126, entitled “Table Adapter with Joint Assembly,” issued Dec. 22, 2015, and hereby incorporated by reference in its entirety. The positioning adapter (100) shown in FIGS. 1 and 2 is interchangeable with the positioning adapter (300). With such a configuration, the skull clamp (10) is connectable to a patient support table by way of its connection with positioning adapter (100), which is connectable with joint assembly (204), which is connectable with the first portion (202) of the table adapter (200), which in turn connects with the patient support structure or table (not shown).

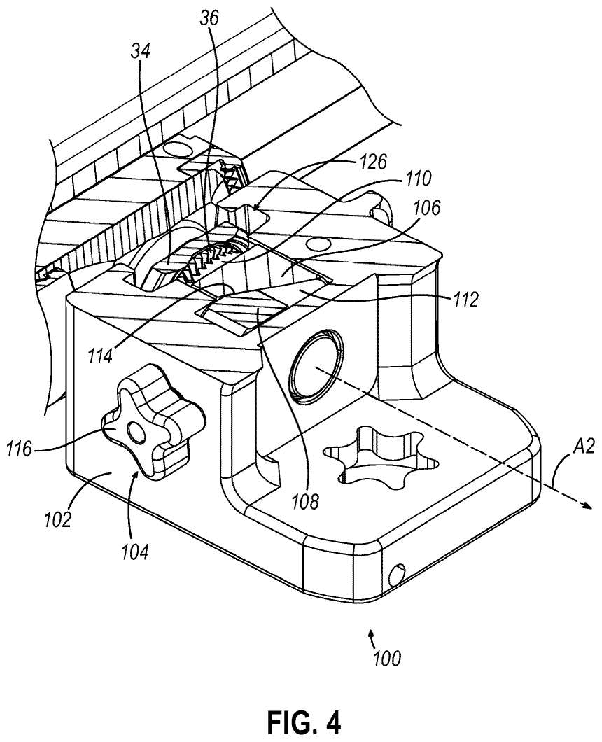

FIG. 4 illustrates the positioning adapter (100) in cross section, with the skull clamp (10) being in the adjustable state. In particular, the positioning adapter (100) includes a lock assembly having a body (102), a knob (104), a lock member (106), and a wedge member (108). The lock member (106) includes a starburst (110) or engaging feature that has a plurality of teeth. As mentioned above, the skull clamp (10) includes connection member (30), and connection member (30) includes a flange (34) that extends from one side of the connection member (30). The flange (34) includes another lock member in the form of a starburst (36) or engaging feature, also with a plurality of teeth. In the present example, the flange (34) is in the form of a circular disk that protrudes from the connection member (30). The circular disk includes a central opening or bore that is configured to receive a bolt or rod structure as described further below. In the configuration shown in FIG. 4, starbursts (110, 36) are disengaged as seen by the gap or space between the respective pluralities of teeth.

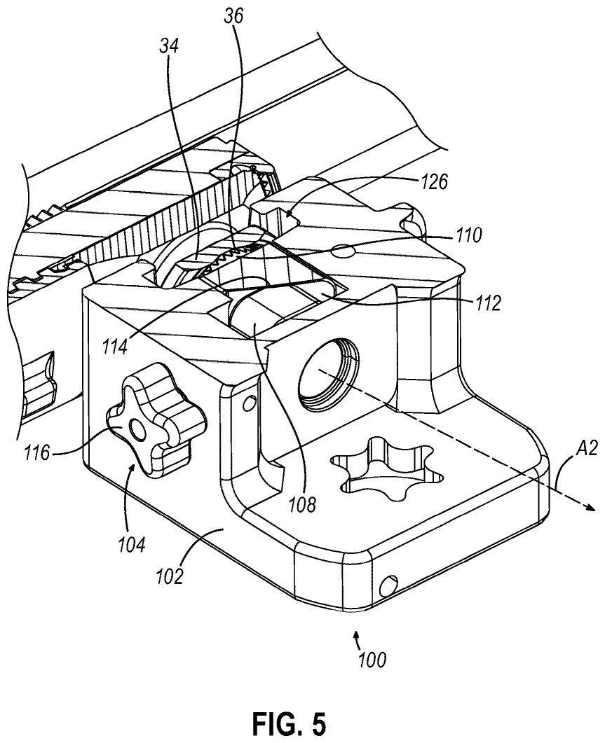

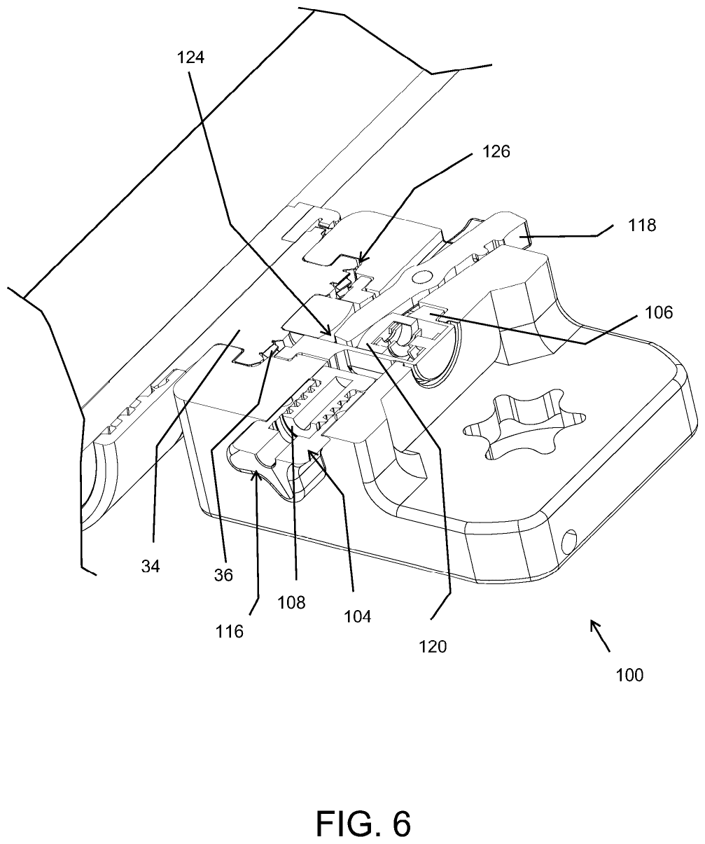

FIG. 5 illustrates a similar view to FIG. 4, except the starbursts (110, 36) are engaged as seen by the lack of a gap or space between the respective pluralities of teeth. To move the position adapter (100) into engagement with the connection member (30) of the skull clamp (10), the knob (104) is rotated. In response to rotation of the knob (104), the wedge member (108) translates along the knob (104) based on a threaded engagement between the knob (104) and the wedge member (108) as best seen in FIG. 6. The direction of rotation of the knob (104) will dictate the direction of translation of the wedge member (108).

As seen in FIGS. 4 and 5, the lock member (106) and the wedge member (108) each have an angled surface (112, 114) and each of these angled surfaces (112, 114) are in contact with one another. As the wedge member (108) translates along the knob (104) in a direction away from a star-shaped end (116) of the knob (104), the angled surface (114) of the wedge member (108) drives the lock member (106) toward the flange (34) of the connection member (30) by the interaction with the angled surface (112) of the lock member (106). When the connection member (30) and the positioning adapter (100) are in the disengaged state where the pluralities of teeth of starbursts (110, 36) are not in contact, the skull clamp (10) may be adjustably rotated about an axis (A2) defined through the center of starbursts (110, 36). This adjustment of the skull clamp (10) can be referred to as adjusting the skull clamp (10) from the one side of the patient to the other side when the patient is in the prone or supine position.

FIG. 6 also shows a perspective cross section view of the positioning adapter (100) and a portion of the skull clamp (10). As shown in FIG. 6, the connection member (30) of the skull clamp (10) and the positioning adapter (100) are in the disengaged state where the starbursts (36, 110) are not in contact. The positioning adapter (100) also includes a bolt (120) that is located within an opening in the lock member (106), and a spring (122) between an end of the bolt (120) and an interior wall of the lock member (106). In this manner, the spring (122) biases the bolt (120) towards the connection member (30). In particular, this bias of the bolt (120) locates an end of the bolt within an opening or recess in the flange (34) such that positioning adapter (100) remains connected with connection member (30) of the skull clamp (10) even when the starbursts (36, 110) are disengaged.

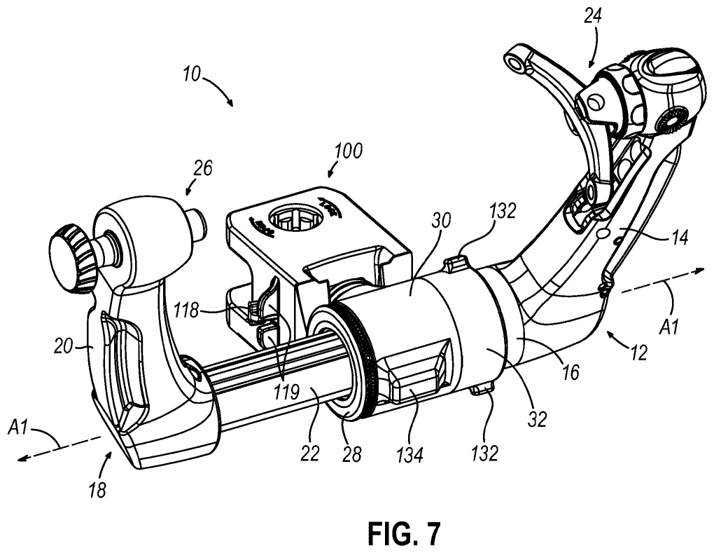

The bolt (120) includes an opening (124) that extends through the side of the bolt (120), and within the opening (124) an end of a release lever (118) is located. When desirable to completely separate the skull clamp (10) from the position adapter (100), release lever (118) is rotated about its pinned connection with the body (102) of the positioning adapter (100). This motion drives the bolt (120) away from the flange (34) and compresses the spring (122) allowing for the end of the bolt (120) to evacuate the opening or recess within the flange (34). A pair of stops (119) as seen in FIGS. 1 and 7 prevent over-rotation of the release lever. With the starbursts (36, 110) disengaged and the bolt (120) moved out of the opening in the flange (34), the skull clamp (10) can be removed from or separated from the positioning adapter (100) by moving the flange (34) of the skull clamp (10) out of a slot (126) defined by positioning adapter (100). In a reverse manner, when installing or connecting the skull clamp (10) to the positioning adapter (100), release lever (118) is again actuated to retract the bolt (120) so that bolt (120) is not obstructing the slot (126) such that the flange (34) of connection member (30) of skull clamp (10) can be positioned within the slot (126).

In the present example as shown in FIGS. 1-6, the engagement between the flange (34) of the connection member (30) and the slot (126) of the positioning adapter (100) enables transferring the weight of the skull clamp (10) as well as the head of the patient positioned within the skull clamp (10) to the table adapter (200) that is connectable with the positioning adapter (100). This weight transfer occurs even when the engaging features or starbursts (36, 110) are in a disengaged or non-contacting state. With this configuration, the surgeon or user is able to steady the skull clamp (10) with minimal or less required force and focus on the orientation or positioning of the skull clamp (10) without also having to physically support the weight of the skull clamp (10) and head of the patient secured within the skull clamp (10). In some versions, this weight supporting feature of the skull clamp (10) and positioning adapter (100) can be figuratively considered like an additional hand or pair of hands that support the weight of the skull clamp (10) and head of the patient.

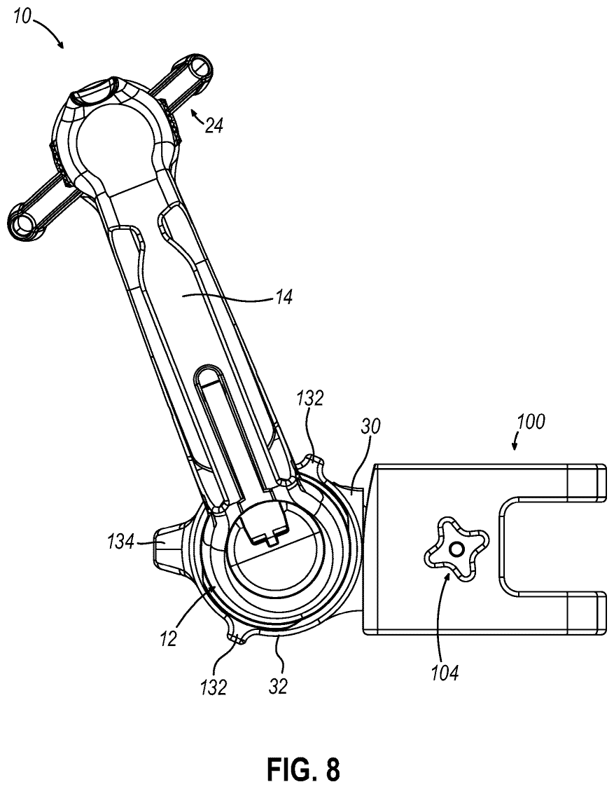

Referring now to FIGS. 7 and 8, the skull clamp (10) is shown rotated to a second rotatable position from the first rotatable position shown in FIGS. 1 and 2. As shown, this rotational adjustment is about the axis (A1) that is defined by the base of the skull clamp (10), or in other words defined by the combined lateral portions (16, 22) of the first and second arms (12, 18) of the skull clamp (10). Rotational adjustment in this manner moves or tilts the skull clamp (10) along the sagittal plane. Moreover, this tilt adjustment is achieved without changing the location of the base of the skull clamp (10) relative to the patient support table or structure that the skull clamp (10) would be connected with as described above.

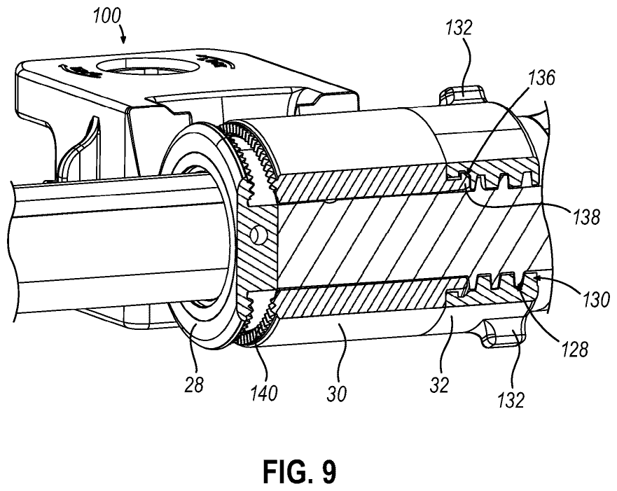

As mentioned above, the skull clamp (10) includes an adjustment assembly that includes the toothed ring (28), the connection member (30), and the lock ring (32). These components work together with the lateral portion (16) of the first arm (12) to provide for this sagittal adjustment. As shown in FIG. 7, in the locked state the first and second arms (12, 18) are not rotatable about the axis (A1) extending through the base of the skull clamp (10). FIG. 9, however, shows the unlocked state where the first and second arms (12, 18) are rotatably adjustable about the axis (A1) extending through the base of the skull clamp (10).

Still referring to FIG. 8, to transition the skull clamp (10) from the fixed state to the adjustable state for the adjustment along the sagittal plane, a user will rotate the lock ring (32). The lock ring (32) has threads (130) and is threadably connected with the lateral portion (16) which also includes corresponding threads (128). The lock ring (32) includes a pair of tabs or projections (132) that can assist with rotation of the lock ring (32). In these examples, the connection member (30) also includes a tab or projection (134). The lock ring (32) also has a groove (136) that is configured to receive a resilient locking tab (138) of the connection member (30). As shown in FIG. 9, the resilient locking tab (138) selectively connects the connection member (30) with the lock ring (32). In this manner when lock ring (32) is rotated, it rotates along the threads (128) of the lateral portion (16) and at the same time moves along the lateral portion (16). The connection member (30), which is not threadably engaged with the lateral portion (16) translates along the lateral portion (16) as lock ring (32) rotates, with the resilient locking tab (138) sliding within the groove (136) yet maintaining the connection between the connection member (30) and the lock ring (32).

As seen in FIG. 9, with this movement described above the connection member (30) separates from the toothed ring (28) as seen by the gap or space between the surface of the toothed ring (28) with the plurality of teeth and the corresponding toothed surface (140) on the end of the connection member (30). It is noted here that the toothed ring (28) remains stationary relative to the lateral portion (16) as it has a pinned connection with the end of the lateral portion (16). With the toothed ring (28), connection member (30), and lock ring (32) in these orientations, the skull clamp (10) is in the adjustable state where the first and second arms (12, 18) can be rotated in unison along the sagittal plane by rotating the first and second arms (12, 18) about the axis (A1) defined by the lateral portions (16, 22), also referred to herein as the base of the skull clamp (10). When rotating the skull clamp (10) to make this adjustment, the lateral portion (16) will slide freely relative to the interior surface of the connection member (30) through which the lateral portion (16) extends. However, the toothed ring (28) and the lock ring (32) will rotate with the first arm (12) as it is adjusted.

Once the desired position is obtained for the adjustment along the sagittal plane, the lock ring (32) is rotated in the opposite direction from that described above so as to move the lock ring (32) along the lateral portion (16) towards the toothed ring (28). Again, based on the connection between the resilient locking tab (138) and the groove (136) of the lock ring (32), the connection member (30) will translate or slide along the lateral portion (16) without rotation as the lock ring (32) rotates. Ultimately, the toothed surface (140) of the connection member (30) will engage with the plurality of teeth on the toothed ring (28), which locks or fixes the rotational position of the skull clamp (10) about the axis (A1).

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

Having shown and described various embodiments of the present invention, further adaptations of the methods and systems described herein may be accomplished by appropriate modifications by one of ordinary skill in the art without departing from the scope of the present invention. Several such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. A device for use in a medical procedure for stabilizing a head of a patient comprising a skull clamp that comprises:
a. a first arm and a second arm, wherein each of the first arm and the second arm have an upright portion;
b. a stabilization assembly connectable with each upright portion, wherein the stabilization assembly is configured to retain a stabilizing feature that is configured to contact the head of the patient;
c. a base defined by one or more lateral portions of a select one or both of the first and second arms, wherein the base defines a first axis; and
d. an adjustment assembly located on the base of the skull clamp, wherein the adjustment assembly is configured to place the skull clamp in a select one of a first fixed state and a first adjustable state, wherein in the first adjustable state the skull clamp is adjustable about the first axis to move at least a portion of the skull clamp along a sagittal plane defined by the patient when in a prone or supine position.

2. The device of claim 1, wherein the adjustment assembly comprises a first locking feature fixed with the base of the skull clamp and a second locking feature selectively moveable along the base of the skull clamp.

3. The device of claim 2, wherein the first locking feature comprises an interference fit with the second locking feature.

4. The device of claim 2, wherein the first locking feature comprises a first plurality of teeth and the second locking feature comprises a second plurality of teeth.

5. The device of any one of claims 2 through 4, wherein the adjustment assembly comprises a third locking feature that is rotatably connectable with the base of the skull clamp.

6. The device of claim 5, wherein the third locking feature is threadably engaged with the base.

7. The device of any one of claims 5 through 6, wherein the third locking feature is rotatable about the base and the second locking feature translates relative to the base upon rotation of the third locking feature.

8. The device of any one of claims 5 through 7, wherein the second locking feature comprises a resilient tab and the third locking feature comprises a groove configured to receive the resilient tab of the second locking feature.

9. The device of claim 8, wherein the resilient tab is configured to freely slide within the groove when the third locking feature is rotated about the base, and the second locking feature is configured to translate along the base upon rotation of the third locking feature relative to the base.

10. The device of any one of claims 5 through 9, wherein the second locking feature includes a tab and the third locking feature includes a tab, wherein the tabs are configured to provide a gripping surface.

11. The device of claim 1, wherein the adjustment assembly is configured to adjust the skull clamp along the sagittal plane while maintaining the relative position of the base of the skull clamp to a table adapter that is configured to connect the skull clamp with a patient support table.

12. The device of claim 1 further comprising a position adapter configured to selectively connect with the skull clamp at a first end, wherein the position adapter is further configured to place the skull clamp in a select one of a second fixed state and a second adjustable state, wherein in the second adjustable state the skull clamp is adjustable about a second axis transverse to the first axis.

13. The device of claim 12, wherein the positioning adapter comprises a slot configured to receive a flange of the second locking feature.

14. The device of claim 13, wherein the positioning adapter comprises a bolt and the flange comprises a recess configured to receive the bolt, wherein the engagement of the bolt with the recess and the slot with the flange maintains a connection between the positioning adapter and the second locking feature with the skull clamp in the second adjustable state.

15. The device of claim 14, wherein positioning adapter comprises a spring configured to bias the bolt into the recess of the flange.

16. The device of any one of claim 14 or 15, wherein the positioning adapter comprises a release lever configured to selectively retract the bolt from the recess.

17. The device of any one of claims 12 through 16, wherein the position adapter comprises a lock assembly having a first lock member and a second lock member, wherein the first and second lock member are selectively engageable such that when engaged the skull clamp is in the second fixed state and when disengaged the skull clamp is in the second adjustable state.

18. The device of claim 17, wherein the first lock member and the second lock member have an interference fit when engaged.

19. The device of any one of claims 12 through 18, wherein the first lock member and second lock member each comprise a plurality of teeth wherein the pluralities of teeth are selectively engageable such that when engaged the skull clamp is in the second fixed state and when disengaged the skull clamp is in the second adjustable state.

20. The device of claim 19, wherein the positioning adapter comprises a wedge member configured to translate the lock member so that the first starburst engages the second starburst of the flange.

21. A device for use in a medical procedure for stabilizing a head of a patient comprising a skull clamp and a position adapter, wherein the skull clamp comprises:
a. a first arm and a second arm, wherein each of the first arm and the second arm have an upright portion;
b. a stabilization assembly connectable with each upright portion, wherein the stabilization assembly is configured to retain a stabilizing feature that is configured to contact the head of the patient; and
c. a base defined by one or more lateral portions of a select one or both of the first and second arms, wherein the base defines a first axis; and
wherein the position adapter is configured to selectively connect with the base of skull clamp, wherein the position adapter is configured to place the skull clamp in a select one of a fixed state and an adjustable state, wherein in the adjustable state the skull clamp is adjustable about a second axis transverse to the first axis.

22. The device of claim 21, wherein the position adapter comprises a slot, and wherein the device comprises a connection member having a flange, wherein the slot of the positioning adapter is configured to receive the flange of the connection member, wherein engagement of the flange and the slot is configured to transfer the weight of the head of the patient to a table adapter connectable with the position adapter.

23. The device of claim 22, wherein the flange includes a first engaging feature, and the position adapter includes a second engaging feature configured to selectively engage with the first engaging feature, wherein the engagement of the flange and the slot is configured to transfer the weight of the head of the patient to the table adapter connectable with the position adapter when the first and second engaging features are disengaged.

24. A method of adjusting a skull clamp along a sagittal plane defined by a patient when in the prone or supine position, the skull clamp having (a) a first arm and a second arm each with an upright portion, (b) a stabilization assembly connectable with each upright portion, (c) a base defined by one or more lateral portions of a select one or both of the first and second arms, (d) an axis defined by the base wherein the axis extends perpendicular to the sagittal plane, and (e) an adjustment assembly connectable with the base where the adjustment assembly includes (i) a first locking feature fixed to a portion of the base, (ii) a second locking feature moveable relative to the base, the second locking feature selectively connectable with the first locking feature, and (iii) a third locking feature threadably connected with the base, the third locking feature connectable with the second locking feature, the method steps comprising:
a. rotating the third locking feature about the base to translate and separate the second locking feature from the first locking feature to place the skull clamp in an adjustable state;
b. rotating the first and second arms of the skull clamp about the axis to a desired position, wherein the rotation moves the skull clamp along the sagittal plane; and
c. rotating the third locking feature about the base to translate and engage the second locking feature with the first locking feature to place the skull clamp in a fixed state where the skull clamp is not adjustable about the axis and along the sagittal plane.

1. A device for use in a medical procedure for stabilizing a head of a patient comprising a skull clamp that comprises:
a. a first arm and a second arm, wherein each of the first arm and the second arm have an upright portion;
b. a stabilization assembly connectable with each upright portion, wherein the stabilization assembly is configured to retain a stabilizing feature that is configured to contact the head of the patient;
c. a base defined by one or more lateral portions of a select one or both of the first and second arms, wherein the base defines a first axis; and
d. an adjustment assembly located on the base of the skull clamp, wherein the adjustment assembly is configured to place the skull clamp in a select one of a first fixed state and a first adjustable state, wherein in the first adjustable state the skull clamp is adjustable about the first axis to move at least a portion of the skull clamp along a sagittal plane defined by the patient when in a prone or supine position.

2. The device of claim 1, wherein the adjustment assembly comprises a first locking feature fixed with the base of the skull clamp and a second locking feature selectively moveable along the base of the skull clamp.

3. The device of claim 2, wherein the first locking feature comprises an interference fit with the second locking feature.

4. The device of claim 2, wherein the first locking feature comprises a first plurality of teeth and the second locking feature comprises a second plurality of teeth.

5. The device of claim 2, wherein the adjustment assembly comprises a third locking feature that is rotatably connectable with the base of the skull clamp.

6. The device of claim 5, wherein the third locking feature is threadably engaged with the base.

7. The device of claim 5, wherein the third locking feature is rotatable about the base and the second locking feature translates relative to the base upon rotation of the third locking feature.

8. The device of claim 5, wherein the second locking feature comprises a tab and the third locking feature comprises a groove configured to receive the tab of the second locking feature.

9. The device of claim 8, wherein the tab is configured to freely slide within the groove when the third locking feature is rotated about the base, and the second locking feature is configured to translate along the base upon rotation of the third locking feature relative to the base.

10. The device of claim 5, wherein the second locking feature includes a tab and the third locking feature includes a tab, wherein the tabs are configured to provide a gripping surface.

11. The device of claim 1, wherein the adjustment assembly is configured to adjust the skull clamp along the sagittal plane while maintaining the relative position of the base of the skull clamp to a table adapter that is configured to connect the skull clamp with a patient support table.

12. The device of claim 1 further comprising a position adapter configured to selectively connect with the skull clamp at a first end, wherein the position adapter is further configured to place the skull clamp in a select one of a second fixed state and a second adjustable state, wherein in the second adjustable state the skull clamp is adjustable about a second axis transverse to the first axis.

13. The device of claim 12, wherein the positioning adapter comprises a slot configured to receive a flange of the second locking feature.

14. The device of claim 13, wherein the positioning adapter comprises a bolt and the flange comprises a recess configured to receive the bolt, wherein the engagement of the bolt with the recess and the slot with the flange maintains a connection between the positioning adapter and the second locking feature with the skull clamp in the second adjustable state.

15. The device of claim 14, wherein positioning adapter comprises a spring configured to bias the bolt into the recess of the flange.

16. The device of claim 14, wherein the positioning adapter comprises a release lever configured to selectively retract the bolt from the recess.

17. The device of claim 12, wherein the position adapter comprises a lock assembly having a first lock member and a second lock member, wherein the first and second lock member are selectively engageable such that when engaged the skull clamp is in the second fixed state and when disengaged the skull clamp is in the second adjustable state.

18. The device of claim 17, wherein the first lock member and the second lock member have an interference fit when engaged.

19. The device of claim 12, wherein the first lock member and second lock member each comprise a plurality of teeth wherein the pluralities of teeth are selectively engageable such that when engaged the skull clamp is in the second fixed state and when disengaged the skull clamp is in the second adjustable state.

20. The device of claim 19, wherein the positioning adapter comprises a wedge member configured to translate the lock member so that the first starburst engages the second starburst of the flange.

21. A device for use in a medical procedure for stabilizing a head of a patient comprising a skull clamp and a position adapter, wherein the skull clamp comprises:
a. a first arm and a second arm, wherein each of the first arm and the second arm have an upright portion;
b. a stabilization assembly connectable with each upright portion, wherein the stabilization assembly is configured to retain a stabilizing feature that is configured to contact the head of the patient; and
c. a base defined by one or more lateral portions of a select one or both of the first and second arms, wherein the base defines a first axis; and
wherein the position adapter is configured to selectively connect with the base of skull clamp, wherein the position adapter is configured to place the skull clamp in a select one of a fixed state and an adjustable state, wherein in the adjustable state the skull clamp is adjustable about a second axis transverse to the first axis.

22. The device of claim 21, wherein the position adapter comprises a slot, and wherein the device comprises a connection member having a flange, wherein the slot of the positioning adapter is configured to receive the flange of the connection member, wherein engagement of the flange and the slot is configured to transfer the weight of the head of the patient to a table adapter connectable with the position adapter.

23. The device of claim 22, wherein the flange includes a first engaging feature, and the position adapter includes a second engaging feature configured to selectively engage with the first engaging feature, wherein the engagement of the flange and the slot is configured to transfer the weight of the head of the patient to the table adapter connectable with the position adapter when the first and second engaging features are disengaged.

24. A method of adjusting a skull clamp along a sagittal plane defined by a patient when in the prone or supine position, the skull clamp having (a) a first arm and a second arm each with an upright portion, (b) a stabilization assembly connectable with each upright portion, (c) a base defined by one or more lateral portions of a select one or both of the first and second arms, (d) an axis defined by the base wherein the axis extends perpendicular to the sagittal plane, and (e) an adjustment assembly connectable with the base where the adjustment assembly includes (i) a first locking feature fixed to a portion of the base, (ii) a second locking feature moveable relative to the base, the second locking feature selectively connectable with the first locking feature, and (iii) a third locking feature threadably connected with the base, the third locking feature connectable with the second locking feature, the method steps comprising:
a. rotating the third locking feature about the base to translate and separate the second locking feature from the first locking feature to place the skull clamp in an adjustable state;
b. rotating the first and second arms of the skull clamp about the axis to a desired position, wherein the rotation moves the skull clamp along the sagittal plane; and
c. rotating the third locking feature about the base to translate and engage the second locking feature with the first locking feature to place the skull clamp in a fixed state where the skull clamp is not adjustable about the axis and along the sagittal plane.
